(19) United States
(12) Patent Application Publication
(10) Pub. No.: US 2007/0250345 A1
(43) Pub. Date: Oct. 25, 2007

(54) ELECTRONIC MEDICAL RECORD SYSTEM, METHOD, AND COMPUTER PROCESS FOR THE TESTING, DIAGNOSIS, AND TREATMENT OF SLEEP DISORDERS

(76) Inventors: James Walker, Farmington, UT (US); Brandon Walker, Farmington, UT (US)

Correspondence Address: James Walker, 1726 Hampton CT, Farmington, UT, 84025, US

(21) Appl. No.: 11/739,664

(22) Filed: Apr. 24, 2007

Related U.S. Application Data
(60) Provisional application No. 60/745,431, filed on Apr. 24, 2006.

(51) Int. Cl. G06Q 10/00; A61B 5/00

(52) U.S. Cl. 705/2; 600/300

(57) ABSTRACT

This is a patient electronic medical record system, method, and computer processes that includes the ability to input demographic information, diagnosis specific questionnaire templates, polysomnographic data, technician observations, patient satisfaction surveys to achieve comprehensive and medical documentation that captures patient data prior to, concurrently, and following polysomnography. The system is enabled for a distributed computing environment including graphical user interfaces, text, and polysomnographic input. All information is stored in a database, which allows integrated summarized output, the development of physician interpretative reports, prescriptions, billing information, and database searches. The program can be Internet web-based with an encrypted connection to a secure server or be part of an integrated wide area network.

This application is a claims benefit of my provisional application No. 60745431 filed 24 Apr. 2007

### FIELD OF THE INVENTION

The present invention relates to an electronic medical record system, method, and computer processes for the testing, diagnosis, and treatment of patients with sleep disorders. Accordingly, this invention involves the fields of programming, informational technology, medicine, and other health sciences.

### BACKGROUND OF THE INVENTION

In the past two decades, it has become recognized that sleep disorders are pervasive; approximately 50 million Americans suffer from sleep disorders including snoring and sleep apnea, narcolepsy, restless legs syndrome, and insomnia. It is estimated that 18 million Americans have sleep apnea and 30-55 million suffer from insomnia. The cost to society and health is substantial. For example, the total cost of insomnia, including treatment, lost productivity, and insomnia related accidents, may exceed 100 billion dollars. Sleep apnea has now been determined to be an independent risk factor for hypertension, heart disease, stroke, and diabetes; appropriate treatment can reduce the risks for these conditions.

With the acknowledgement of the importance of sleep, there has been the development of new discipline of sleep medicine. This field has recently been recognized by the American Board of Medical Specialties, the pre-eminent entity overseeing physician certification in the United States. To perform diagnostic testing, there has also been the parallel development of sleep disorders centers, which has become the standard in most major hospitals. In addition and due to demand, there has been proliferation of free-standing sleep disorders centers.

There is multitude of information that goes into the diagnosis and treatment of sleep disorders, which comprises the patient's medical record. This can include a physician examination, medical and sleep history as well as extensive paper and pencil questionnaires. The cornerstone of diagnostic testing is the polysomnogram, a nighttime sleep study conducted under observation in a sleep laboratory. In addition, implementation of therapy is also conducted in conjunction with the polysomnogram (sleep study). An immense amount of data is derived from the analyzed polysomnogram and is generally summarized (4-5 pages) to aid the physician's interpretation of the study. In addition, there are extensive observations made during the course of testing by the attending technician. Pre-sleep and post-sleep surveys are also common. The above paperwork is compounded by situations where studies will be conducted during a diagnostic polysomnogram followed by a second night where therapy is assessed with another polysomnogram. So it is no uncommon for a single patient to have 30-50 pages of information and documentation of their sleep disorder. This information is synthesized into a final report with treatment recommendations by the interpreting physician. A publication of the American Academy of Sleep Medicine, Practice parameters for the indications of polysomnographic procedures: an update for 2005, Sleep, 505-519, 2005, provides both essential items and recommended items to be included in the final report for polysomnography. These items are derived from sources outlined above; i.e., medical examination, patient questionnaires, technician observations and the polysomnogram. The information is not integrated and can reside in a variety of forms, formats, and locations.

The present state of art where the medical record is predominantly based upon paper charts is cumbersome, inefficient, lacks ease of rapid review and accessibility of information; particularly where vast amounts of data have to be reviewed and integrated.

### DETAILED DESCRIPTION

### A. Definitions

In describing and claiming the present invention, the following terminology will be used in accordance with the definitions set for below.

The singular forms “a,” “an,” and, “the” include plural referents unless the context clearly dictates otherwise. Thus, for example, reference to “a patient” includes reference to one or more of such patients, and reference to “a physician” includes reference to one or more physicians.

As used herein the tern “polysomnogram” refers to a comprehensive recording of the biophysiological changes that occur during sleep. This diagnostic test monitors many body functions including brain (EEG), eye movements (EOG), chin muscle activity (EMG), leg muscle activity, heart rhythm (ECG), breathing function by respiratory effort and respiratory airflow, and arterial oxygen saturation. Polysomnography is used to diagnose many types of sleep disorders including narcolepsy, restless legs syndrome, REM behavior disorder, parasomnias, and sleep apnea.

As used herein, the term RTF refers to a rich text format files, which is a standard formalized by Microsoft Corporation for cross-document interchange and is used in specifying formatting of documents. Most word processors are able to read and write RTF documents.

As used herein the term “NIH Rest Legs Syndrome Criteria” refers to guidelines developed by the National Institutes of Health (www.ninds.nih.gov/disorders/restless_legs/detail_restless_legs.htm).

As used herein the term “Epworth Sleepiness Scale” refers to an eight-item scale that is a widely used questionnaire evaluating a patient's general level of daytime sleepiness. Johns, Murray W. (1991). A new method for measuring daytime sleepiness: the Epworth Sleepiness Scale”. Sleep 1991 (14): 540-5.

As used herein the term “Berlin Questionnaire” refers to a 10-question survey that identifies the risk for sleep apnea syndrome (Netzer N C, Stoohs R A, Netzer C M, Clark K, Strohl K P. Using the Berlin Questionnaire to identify patients at risk for the sleep apnea syndrome. Ann Intern Med 1999;131:488).

### B. The Invention

Accordingly, the present invention provides methods and computer processes for integrated electronic medical record of information in the testing, diagnosis, and treatment of sleep disorders. The method allows for computer input of information in a user-friendly format eliminating the need for paper records. Specifically, demographic information, questionnaire information including validated predictive measures for some sleep disorders, pre-sleep questions before polysomnography, detailed technician notes and observations during polysomnography, post-sleep questions, and patient satisfaction survey following polysomnography is directly entered into a relational database through the use of a user-friendly program. The above measures can be entered either by keyboard or a wireless graphic interface such as a tablet computer with touch screen capabilities. In addition, collected analyzed and summarized polysomnographic data form the sleep study can be entered via keyboard or uploaded from RTF files generated by digitally acquired polysomnographic data.

The platform for the electronic medical record system is built upon is event driven programming language with graphical user interface consisting of windows, menus, radio buttons, check boxes, drop-down menus, and icons, and employs a pointing device such as a mouse, trackball, or touch screen in addition to a keyboard. The programming language allows construction and access to databases, which can be managed and queried using a database management system. In addition, all data entered is retained in its original format and can be assessed through the electronic medical record system with patient search/select features, which allows fields and screens to be populate as originally entered. The scope of the present invention is not limited to specific programming language, nor is it in any way limited to by specific database system.

The program can be Internet web-based with an encrypted connection to a secure server or be part of an integrated wide area network.

1. Information Input

The initial data screen consists of patient demographic information, which can be entered either by healthcare personnel or the patient. Similarly, the Health/Sleep Survey data, the next series of screens, is entered by the patient, or in the case of a patient that has difficulty with computer use, transcribed from a completed patient questionnaire. The Health/Sleep survey consists of questions answered by checking appropriate conditions in a checkbox or selecting multiple choice conditions available through a series of drop-down menus. These items are designed to identify symptoms and signs of a variety of sleep disorders. Many of these are evidenced-based surveys or questions such as the NIH restless legs symptom criteria. In addition, validated surveys such as the Epworth Sleepiness Scale and the Berlin Questionnaire are incorporated into the question set. Other information includes bedtime, rise time, time to fall asleep, number of awakenings, and specific sleep-related complaints are polled. Daytime functioning is also assessed. Medical history, including medication use, surgeries, and medical conditions diagnosed by a physician are collected. Additional validated survey instruments can be incorporated into the assessment process, as developed, and are not limited to the Berlin Questionnaire, Epworth Sleepiness Scale, NIH restless legs criteria. A review of symptoms checklist, can also be incorporated in the patient.

Just prior to polysomnography, a computer bedtime questionnaire in the above described user friendly format assessing daytime events, medications taken, and pain level, can be administered. Information derived from this instrument can be essential in interpreting polysomnographic results. For example, almost all medications affect sleep patterns but in different ways and many medications can suppress respiratory drive. Similarly, a morning questionnaire can be administered to assess the patient's perception of sleep quality, length, and compare to normal sleep quality. This information can be important in evaluating the patient's perception of sleep to objective measures.

Another embodiment of the present invention is the ability to capture information during the course of polysomnographic study; documentation of the study and observations from the technologists are essential in interpreting the results. These observations cover a wide range activity. For example, patient behavior (sleep position, abnormal motor activity, behavior, snoring presence and intensity, etc) and physiological (electrocardiographic rhythm) events are critical information in understanding an underlying sleep disorder. All of this information can be captured from the technologist's input data program which is based upon on-screen menus, selectable timed required input, and decision trees useful in implementing therapy. Another feature of this aspect of the program is the ability to send encrypted email alerts (HIPPA compliant) to the interpreting physician or lab director when sentinel events are noted. For example, if a life-threatening heart rhythm such as ventricular tachycardia is present, an email alert can be automatically sent to appropriate personnel so notification of the referring physician can be taken the following day. Evaluation of technical quality of EEG waveforms, respiratory measures, and other physiological variables are noted throughout the night. As with questionnaire all of the information derived from technologist input stored in the database.

Another embodiment of the present invention is the ability to ensure constant notation by the technologist throughout the night and also to covertly monitor technologist's vigilance during the polysomnogram. This is accomplished by means of a timed pop-up window, superimposed on the computer polysomnograph screen window requesting information such as body position, snoring level, arterial oxygen saturation, etc. This information is stored as well as the elapsed time between screen appearance and closing of the window. The elapsed time measures vigilance of the attending technologist.

Following polysomnography, the program allows implementation of patient satisfaction through a survey instrument. Integration of quality measures within the data base can also be accomplished. Such information is essential for continuous quality improvement and is necessary to meet sleep laboratory/center accreditation standards of the American Academy of Sleep Medicine as well as the Joint Commission on Accreditation of Healthcare Organizations (JCAHO).

In addition to the capability to capture patient information prior to, during, and following polysomnography, there is also the means to input information following these processes and includes information for prescriptions, certificates of medical necessity for oxygen and other therapies, diagnoses, ICD-9 codes, CPT codes, and billing information.

2. Information Storage

In another aspect, the present invention allows the user interface program to reside on any number of computers but information entry is stored in a database on a single server on a distributed network system. This allows access simultaneously by many users. Consequently several patients can be accommodated at the same time. In addition, the network system can have redundant storage (mirrored drives) and also can be backed up on a regular basis, ensuring integrity of data. In a reverse fashion, data can be populated from the database for any patient to recapture their responses to questions; i.e., for any patient, there are approximately 200-300 data points. This data can be accessed from any computer that has network drive privileges and the user interface program. For example, a physician can access patient responses in his/her office located remotely from the sleep disorders center as long as the network system is in place. In this manner, all data is available electronically and integrated across all inputs including demographic information, sleep/health survey, bedtime and morning questionnaire, and technologist observations, and summarized polysomnographic data.

3. Report Generation

One aspect of the present invention is the capability to generate a wide variety reports after all data is collected and in a variety of formats. As an example but not limited to this specific word processor, data can be formatted and presented in an automated Microsoft Word document. Another aspect of the medical record system is that data can be integrated across the various collection instruments. As an example, sleep history and indications of disorders can be collated with polysomnographic results and integrated into the physician interpretive report. Although the physician report is tailored to specific a patient, there are essential features that are required. One of the features of the program is that selectable data can be produced in a manner most useful. Therefore, if selected, only pertinent data is displayed and non-essential information ignored. This has the effect of summarizing substantial information to only that which is meaningful. Thus, a 100-item questionnaire can be reduced to only those items that are outside of normal limits or meaningful from a physician's perspective.

Another embodiment of the program is the capability to generate major components of the physician interpretative report. Although the interpretative physician report can vary, the following often comprises elements of such a report: Patient Demographic Information, Reason for Study, Clinical Information, Study Protocol, Clinical Observations, Electrocardiographic Observations, Electrophysiological Measures, Respiratory Measures, Conclusions (can be named Findings, Impressions, or similar description), and Recommendations (can be called Treatment Plan or similar description). With the exception of Recommendations, most of the information contained in the report is derived from the various program inputs. The elements of the interpretative report are user selectable and can be customized to meet the requirements of the interpreting physician. For example, items on the Clinical Summary can be can be selected from a checklist by the interpreting physician to include age, sex, presenting symptoms, daytime functioning, physical characteristics, (weight, height, body mass index, airway classification, and neck circumference), medical history, and medications). This same principle of user-selection applies to each section of the interpretative report. There are also options available during the report generation in the Conclusions and Recommendation sections. Once the draft report is generated word processor format (e.g., Microsoft Word), it can by edited or enhanced by computer keyboard, tablet pen, or verbally through voice recognition software.

4. Searchable Database

Also, in addition to the information collection and report generation capability of the present invention, there is the capability to perform a wide variety of searches of the database for a number of reasons, including but not limited to continuous quality improvement, research, and marketing.

It is to be understood that the above described process and modes of application are only illustrative of preferred embodiments of the present invention. Numerous modifications and alternative arrangements may be devised by those skilled in the art without departing from the spirit and scope of the present invention. Thus, while the present invention has been described above with particularity and detail in connection with what is presently deemed to be the most practical and preferred embodiments of the invention, it will be apparent to those of ordinary skill in the art that numerous modifications, including but not limited to function and manner of operation may be made without departing from the principles and concepts set forth herein.

### FIGURES

FIG. 1 shows some of computer inputs to the network database system, which includes Physician Information, Sleep/Health Survey, Technician Observations, Analyzed Polysomnographic Data, Bedtime and Morning Questionnaire.

FIG. 2 lists examples of information gathered by the Sleep/Health Survey completed by the patient either on-line, over a secure web-based Internet connection, or transcribed by office staff form a pencil/paper patient filled out survey. Other information not noted would include demographic data, insurance information, address, social status, etc.

FIG. 3 indicates some variables collected during polysomnographic data acquisition by the sleep technologist.

FIG. 4 shows additional information that is entered into the program ans stored network database system.

FIG. 5 shows the integration of all information to derive the Physician Report.

FIGS. 6-8 illustrates an example of the Physician Report derived from information on the integrated database.

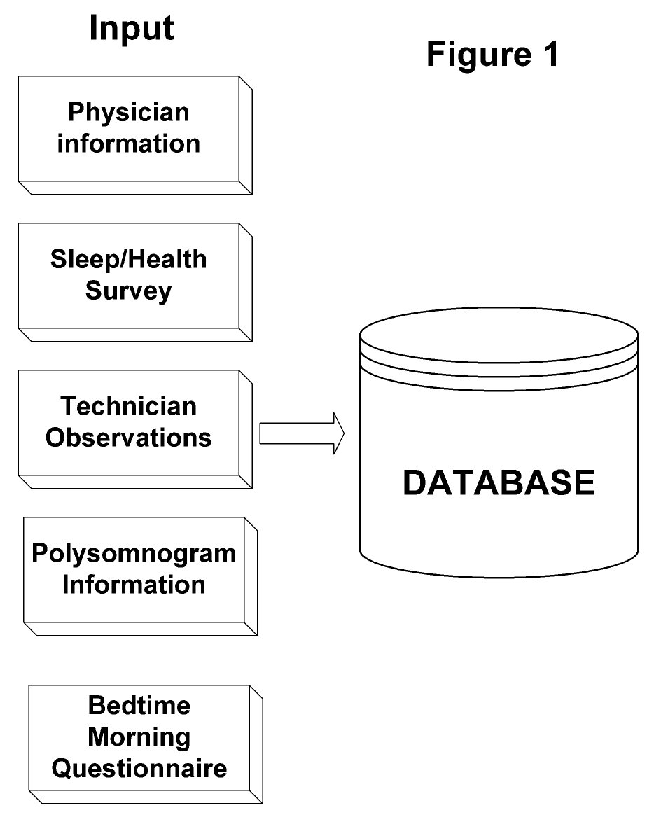

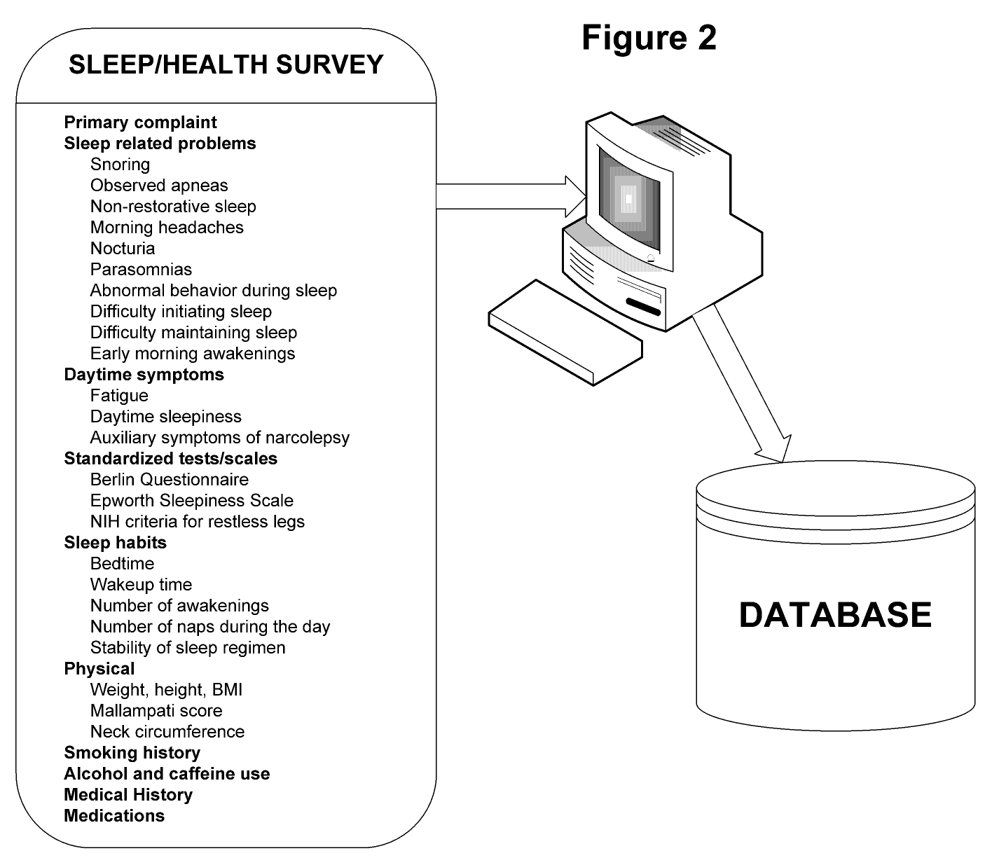

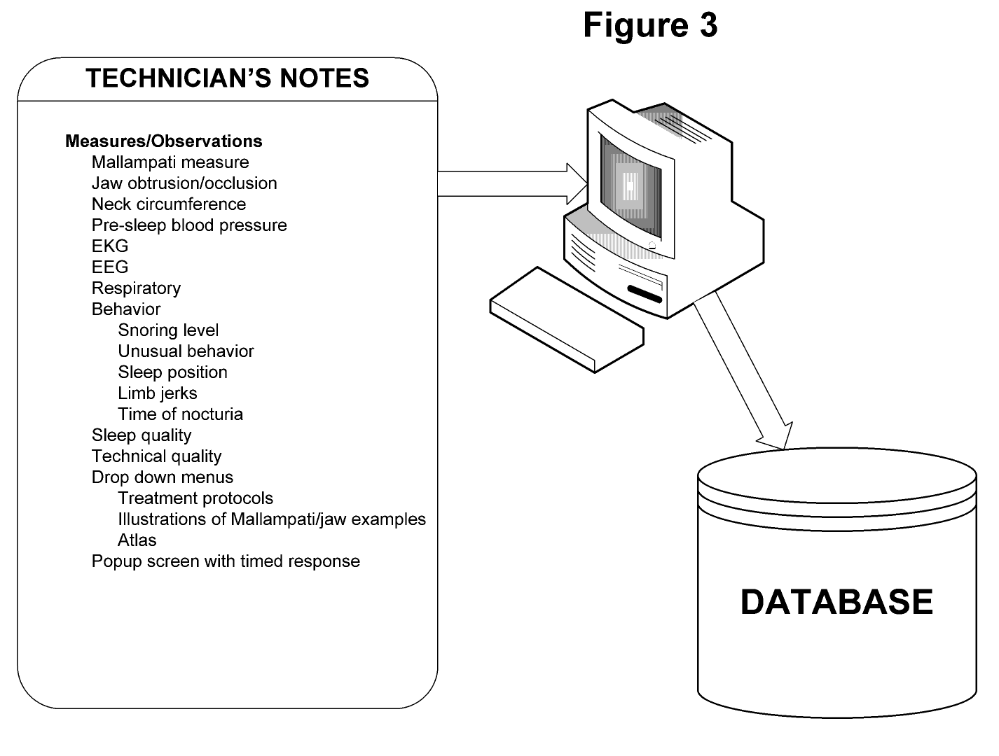

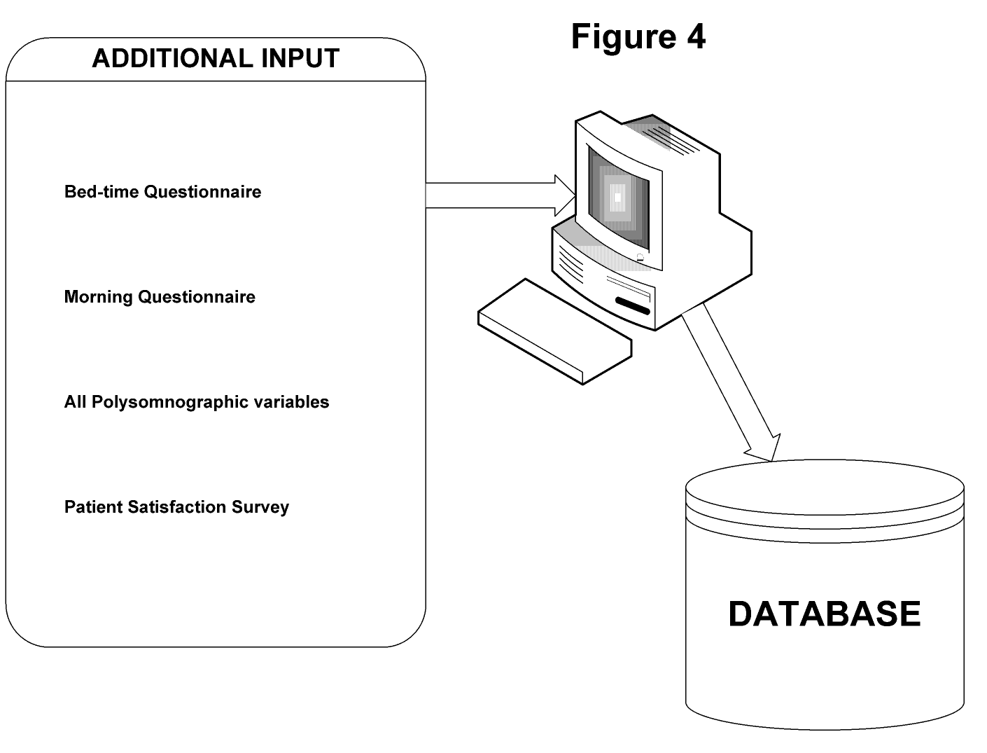

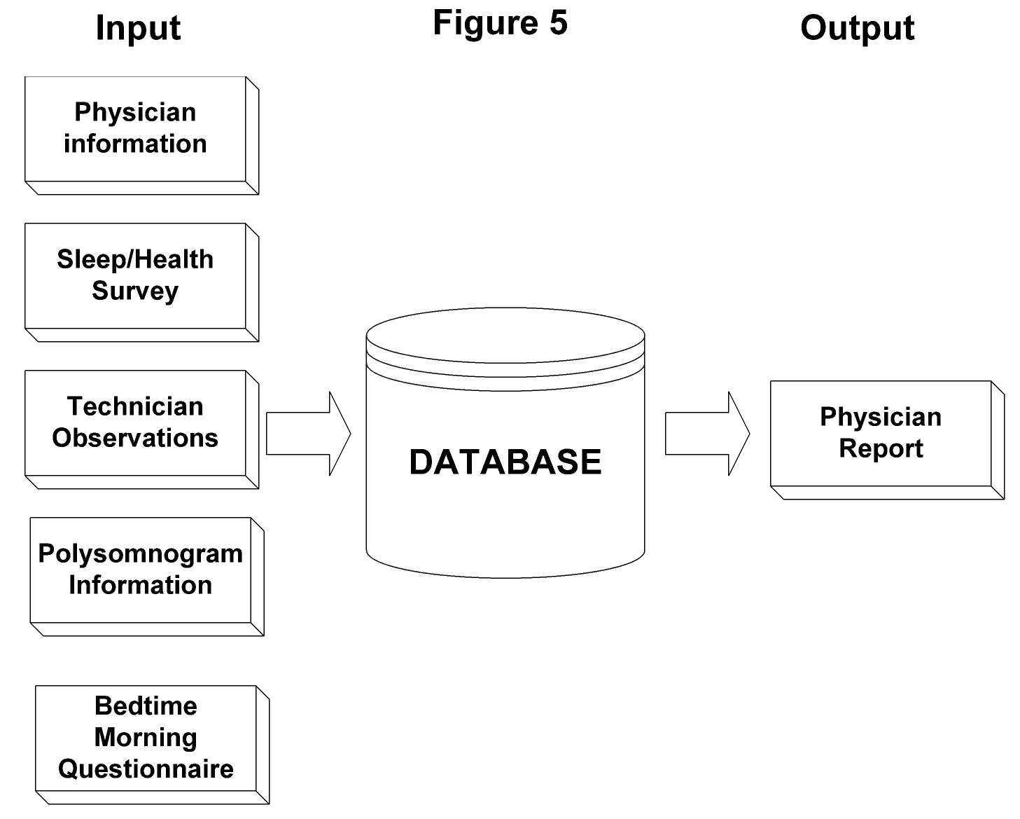

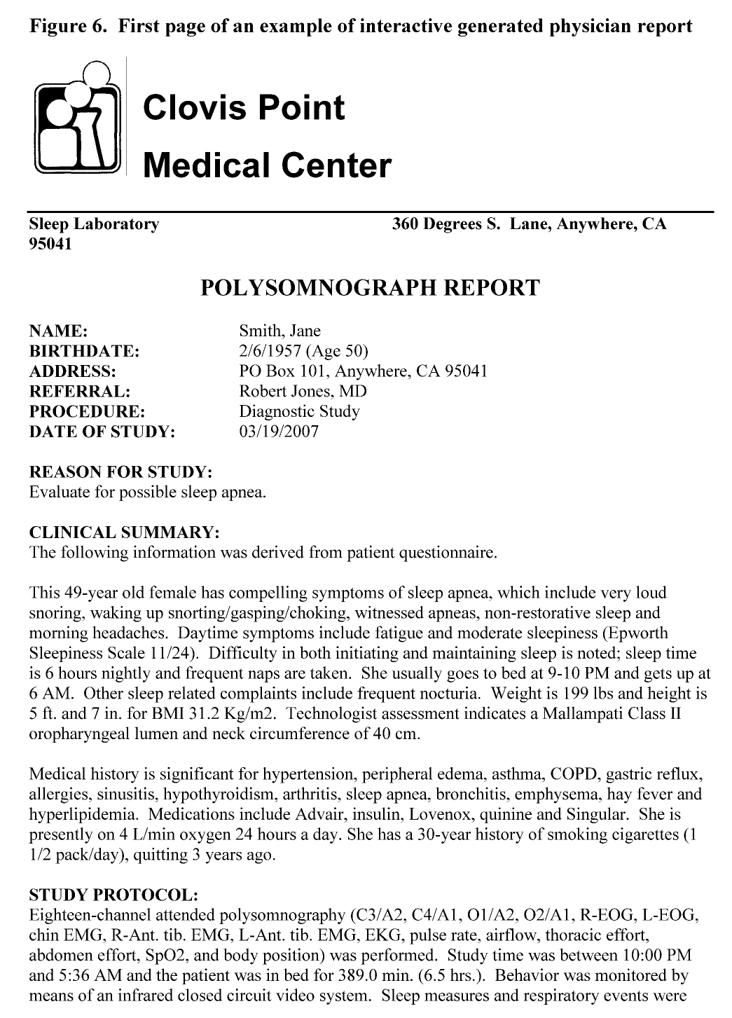

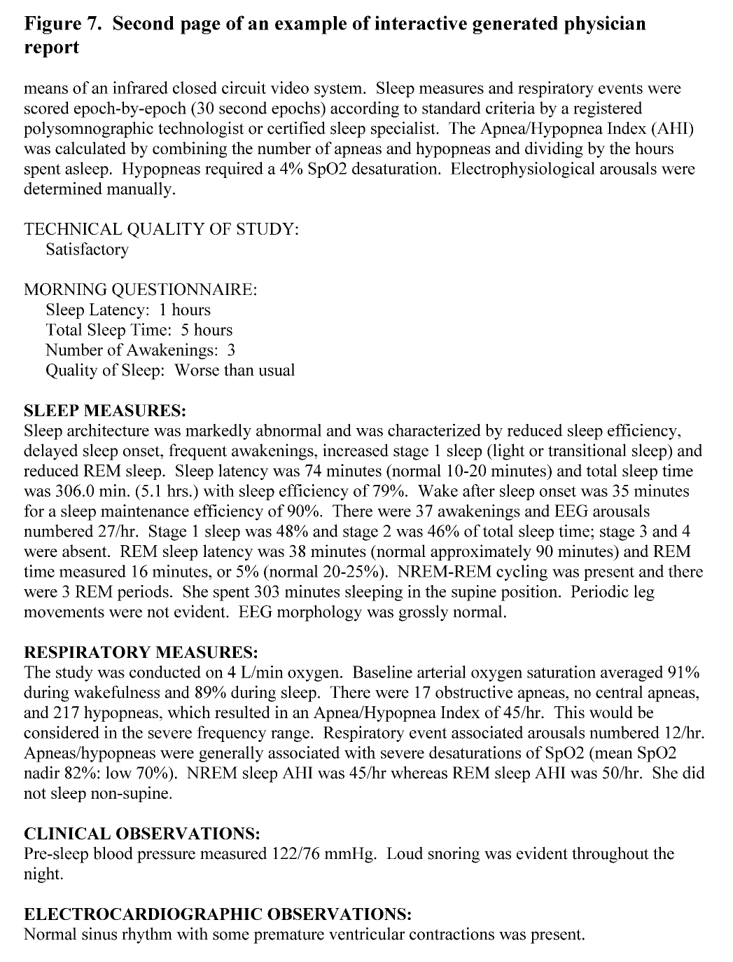

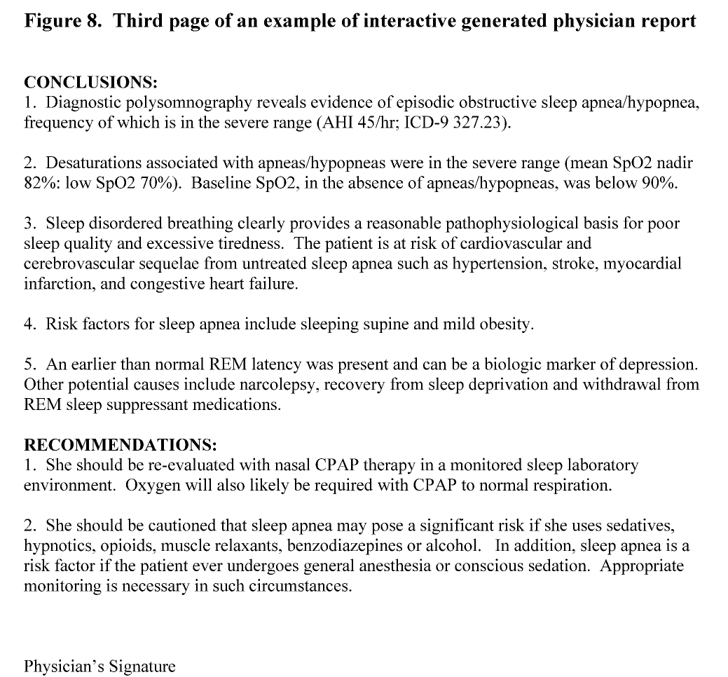

What is claimed is

1. An electronic medical record system, method, and computer process for the diagnosis and treatment of sleep disorders that allows collection of physician examination, patient sleep/health survey, polysomnographic data, technician observations, and patient satisfaction survey information, which is stored in a network searchable database and is used to generate summary information, technician reports, and physician interpretative report.

2. A system as in claim 1 where the platform for the electronic medical record system is built upon is event driven programming language with graphical user interface consisting of windows, menus, radio buttons, check boxes, drop-down menus, and icons using a pointing device such as a mouse, trackball, touch screen, computer keyboard, graphic interface, free text, and/or verbally through voice recognition software and can be Internet web-based with an encrypted connection to a secure server or be part of an integrated wide area network.

3. A system as in claim 2 where the programming language allows construction and access to databases, which can be managed and queried using a database management system.

4. A system as in claim 2 where all data entered is retained in its original format and can be assessed through the electronic medical record system with patient search/select features, which allows fields and screens to be populate as originally entered.

5. A system as in claim 1 where patient information can be entered by physician, medical staff, patient, or sleep technologist and is stored on in a database on an integrated network system.

6. A system as in claim 5 where information from a physician history and physical can be electronically and stored on a database on an integrated network system.

7. A system as in claim 5 where patient responses to a Sleep/Health Survey can be entered by patient, either on-line, over the internet on a secure server, or transcribed by medical staff from paper documents and is stored on in a database on an integrated network system.

8. A system as in claim 1 where analyzed polysomnographic summary data can be imported by various computer file formats to the database or directly entered by medical staff.

9. A system as in claim 1 where technologist's observations are entered during the course of polysomnography.

10. A system as in claim 9 where during polysomnographic acquisition, technician comments, observations, and responses to checkbox items are computer entered and stored in database and form a subset of patient information.

11. A system as in claim 9 whereby during polysomnographic data acquisition, the technician is prompted to input information by a screen popup display, that the elapsed time between screen popup display and response to the query on the display is stored in the database systems as a means to covertly monitor technician vigilance.

12. A system as in claim 9 where during polysomnographic data acquisition, technician support can be provided in terms of on-line atlas of various EKG, respiratory, EEG patterns, and treatment protocols.

13. A system as in claim 9 where there is the ability to automatically send encrypted email alerts (HIPPA compliant) to the interpreting physician or lab director when sentinel events observed during polysomnographic data acquisition, such as seizures, exceptionally low arterial oxygen saturation, or dangerous electrocardiographic rhythms are noted by the sleep technologist.

14. A system as in claim 1 where an on-line or web-based patient satisfaction survey can be completed as a means to assess and improve quality of patient care.

15. A system as in claim 1 where interactive interpretative physician report is generated from both knowledge database and integrated information derived from subsets of information consisting of physical examination, Sleep/Health Survey, polysomnographic data, technologist observations.

16. A system as in claim 15 where the physician interpretative report is comprised of interactive with drop down menus, multiple options for declarative statements, different formatting for sentence structure, free text entry, and the ability to edit all aspects of the report during generation.

17. A system as in claim 15 where the physician interpretative report can be modified by customized section headings, sections to include in report, variables to be included within each section, order of presentation, and wording formats of sentences.

16. A system as in claim 15 where the physician interpretative report is generated in a word processing report and thereby eliminates the need for dictation and transcription.

17. A system as in claim 15 where patient diagnosis developed in the physician interpretative report is directed by a knowledge base comprising practice standards and model of care algorithms.

18. A system as in claim 15 where patient treatment developed in the physician interpretative report is directed by a knowledge base comprising practice standards and model of care algorithms.

19. A system as in claim 15 where the physician interpretive report requires less time to complete because decisions are assisted by a knowledge base comprising practice standards and model of care algorithms and lack of time spent on dictation and transcription.
